(19) United States
(12) Patent Application Publication
(10) Pub. No.: US 2024/0293605 A1
(43) Pub. Date: Sep. 5, 2024

(54) Device for Removal of Implants and Associated Method of Use

(71) Applicant: Bateman Bottle, LLC, Wilmington, DE (US)

(72) Inventors: Michael Bateman, Highland Ranch, CO (US); Andy Edward Denison, Temecula, CA (US); Peter John D'Aquanni, Murrieta, CA (US); Jon Jensen, Vanderbilt, MI (US)

(21) Appl. No.: 18/661,622

(22) Filed: May 11, 2024

Related U.S. Application Data
(63) Continuation-in-part of application No. 16/645,767, filed on Mar. 9, 2020, now Pat. No. 11,980,394.
(60) Provisional application No. 62/515,314, filed on Jun. 5, 2017.
(60) Provisional application No. 63/507,287, filed on Jun. 9, 2023.

(51) Int. Cl. A61M 1/00

(52) U.S. Cl. CPC A61M 1/741; A61M 2202/0014; A61M 2202/06; A61M 2205/3331

(57) ABSTRACT

In one aspect, an implant removal device for removal of ruptured silicone breast implants includes a hollow container having a middle portion disposed between a first end and a second end. The device also has a connector port coupled to the second end that is coupled to a suction device during use. The device also includes a nozzle coupled to the first end that is configured for placement against an incision of a patient for removing a ruptured breast implant from the patient. The middle portion of the hollow container defines one or more vent holes extending between an interior and exterior container surface with the one or more vent holes configured to reduce negative pressure within the hollow container when the suction device is activated during the removal process.

### CROSS-REFERENCE TO RELATED APPLICATION

This application is a continuation-in-part application of U.S. Application No. 16,645,767, filed on Mar. 9, 2020, entitled “Device for Removal of Implants and Associated Method of Use,” which claims priority to PCT/US2018/036106 filed on May 6, 2018, and claims the benefit of U.S. Provisional Application Ser. No. 62/515,314, filed on Jun. 5, 2017. This application also claims the benefit of U.S. Provisional Application Ser. No. 63/507,287, filed on Jun. 9, 2023, entitled “Removal Device With Vent Holes And Associated Method of Use.” All these applications are incorporated herein by reference for all purposes.

### TECHNICAL FIELD

This disclosure is directed, in general, to a device used to remove implants from a patient, and typically to devices and methods for the removal of silicone breast implants.

### BACKGROUND

The following discussion of the background is intended to facilitate an understanding of the present disclosure only. It should be appreciated that the discussion is not an acknowledgement or admission that any of the material referred to was part of the common general knowledge at the priority date of the application.

A breast implant is a prosthesis used to change the size, shape, and contour of a human breast. Breast implants are implanted under the breast tissue or under the chest muscle to increase breast size (augmentation), to rebuild breast tissue after mastectomy or other damage to the breast (reconstruction), or to correct congenital defects in the chest wall. They are also used in revision surgeries, which correct or improve the result of an original surgery.

Breast implants are available in many different forms. There are two types of breast implants approved for sale in the United States: saline-filled breast implants (i.e., saline breast implants), in which an implant shell is filled with sterile salt water, and silicone-filled breast implants (i.e., silicone breast implants), in which the implant shell is filled with a silicone gel.

Both types have a silicone outer shell. They vary in size, shell thickness, shell surface texture, and shape (contour).

Ruptured silicone breast implants can result after placement into the human body. A rupture is a tear or hole in the outer shell of the breast implant. When this occurs in a saline breast implant, it deflates, meaning the saltwater (saline) solution leaks from the shell. Silicone gel is thicker than saline, so when a silicone gel-filled implant ruptures, the gel may remain in the shell or in the scar tissue that forms around the implant (intracapsular rupture). Silicone gel that leaks outside the capsule surrounding the implant may travel (migrate) away from the breast.

The leaked silicone gel may cause lumps to form in the breast or in other tissue, most often the chest wall, armpit or arm. It may be difficult or impossible to remove silicone gel that has traveled to other parts of the body. Ruptured silicone breast implants can cause breast pain or changes in the contour or shape of the breast. Accordingly, the FDA (Food and Drug Administration of the United States) recommends removing both saline-filled and silicone gel-filled breast implants if they have ruptured.

In many instances, it is desirable to remove the ruptured silicone breast implant and leaking silicone gel. Removal after rupture is a time consuming, tedious and difficult process. Typically, such removal is performed by manual extraction utilizing surgical sponges.

The present disclosure provides a simple, efficient device and associated method to address this problem. The device of the present disclosure is also appropriate for the removal of other types of implants from a patient, including un-ruptured silicone breast implants, saline breast implants, and buttocks implants and the like that include an outer shell that is filled with saline or silicone gel or another filling material.

### SUMMARY

According to an illustrative embodiment, a removal device for removing an implant by an operator is presented. The removal device includes a hollow container extending between a first end and a second end along a longitudinal axis. A middle portion is disposed between the first and second ends. The hollow container has an interior container surface and an opposing, exterior container surface with the interior container surface defining a volume for receiving and containing the implant. The removal device further includes a connector port coupled to the second end of the hollow container and adapted for connection to a suction device. A nozzle is coupled to the first end of the hollow container, extending away from the first end along a nozzle axis. The nozzle has an interior nozzle surface and terminates at a nozzle opening. The hollow container defines a set of vent holes extending between the interior and exterior container surfaces with the set of vent holes configured to reduce negative pressure within the hollow container when the suction device is activated.

According to another illustrative embodiment, a removal device for removing an implant is presented. The removal device includes a hollow container extending between a first end and a second end along a longitudinal axis. A middle portion is disposed between the first and second ends. The hollow container has an interior container surface defining a volume for receiving and containing the implant. The removal device further includes a connector port coupled to the second end of the hollow container and adapted for connection to a suction device. A nozzle, having a cylindrical body, is coupled to the first end of the hollow container, extending away from the first end along a nozzle axis. The nozzle has an interior nozzle surface and terminates at a nozzle opening. The nozzle axis is offset from the longitudinal axis such that the nozzle opening is offset from the middle portion of the hollow container along the longitudinal axis for aligning with the implant during a removal process. The hollow container further includes a first plurality of vent holes positioned through the middle portion and a second plurality of vent holes positioned through the middle portion on an opposing side from the first plurality of vent holes. The first and second plurality of vent holes are configured to reduce negative pressure within the hollow container when the suction device is activated.

According to another illustrative embodiment, a method for removing an implant through an incision in skin of a patient using a suction device and a removal device is presented. The removal device includes a hollow container extending between a first end and a second end along a longitudinal axis. A middle portion is disposed between the first and second ends. The hollow container further includes an interior container surface and an opposing, exterior container surface with the interior container surface defining a volume. A nozzle is coupled to the first end of the hollow container. The nozzle extends away from the first end, wherein the hollow container defines one or more vent holes extending between the interior and exterior container surfaces. The method comprises the steps of coupling the suction device to the second end, grasping the hollow container with one hand of the operator, and inserting the removal device within the incision such that the nozzle opening is in proximity to the implant. The method further comprises the steps of activating the suction device to create a negative pressure within the volume of the hollow container, and adjusting the negative pressure to a desired negative pressure within the volume of the hollow container by adjusting the position of the one hand of the operator to uncover, partially cover or fully cover the one or more vent holes, with the desired negative pressure being a negative pressure from a minimum negative pressure to a maximum negative pressure. The method further comprises the step of drawing the implant from the patient through the nozzle and into the volume of the hollow container at the desired negative pressure. Other embodiments are disclosed below.

### DESCRIPTION OF THE DRAWINGS

Illustrative embodiments of the present inventions are described in detail below with reference to the attached drawing figures, which are incorporated by reference herein and wherein:

FIG. 1A is a schematic, perspective view of an implant removal device according to one illustrative embodiment;

FIG. 1B is a schematic, perspective view of an implant removal device according to another illustrative embodiment;

FIG. 2 is a schematic, perspective view of the implant removal device of FIG. 1A wherein the first end is coupled to the middle portion of the hollow container;

FIG. 3 is a close-up perspective view of the nozzle of FIG. 2;

FIG. 4 is a schematic, section view of an implant removal system including the implant removal device of FIG. 2 prior to coupling the suction device;

FIG. 4A is a close-up section view of the connector port of FIG. 4;

FIG. 5 is a schematic, section view of an implant removal system including the implant removal device of FIG. 2 after coupling the suction device;

FIG. 6 is a schematic, partial section view of the use of the implant removal system of FIG. 5 removing an implant from a patient in an operating room;

FIG. 7 is a schematic, partial section view of the implant removal system after removal of an implant in which the shell and gel of the implant is contained within the hollow container;

FIG. 8A is a perspective view (partially exploded) of the implant removal device according to one illustrative embodiment;

FIG. 8B is a perspective view of the first end of the implant removal device according to another illustrative embodiment;

FIG. 9 is a perspective view of the implant removal device of FIG. 8A wherein the first end is coupled to the middle portion of the hollow container;

FIG. 10 is a close-up perspective view of the nozzle of FIG. 9;

FIG. 11 is a section view of an implant removal system including the implant removal device of FIG. 9 prior to coupling the suction device;

FIG. 11A is a close-up section view of the connector port of FIG. 11;

FIG. 12 is a section view of an implant removal system including the implant removal device of FIG. 9 after coupling the suction device;

FIG. 13 is a perspective view of an implant removal system including the implant removal device of FIG. 9 after coupling the suction device;

FIG. 14 is a perspective view of FIG. 13 of the use of the implant removal system of FIG. 13 in removing an implant from a patient in an operating room in which the user has uncovered at least some of the plurality of vents on one side of the hollow container with the index finger of one hand;

FIG. 15 is a perspective view of FIG. 13 of the use of the implant removal system of FIG. 13 in removing an implant from a patient in an operating room in which the user has partially or fully covered a portion of the plurality of vents on one side of the hollow container with the index finger of one hand;

FIG. 16 is a rotated perspective view of FIG. 13 of the use of the implant removal system of FIG. 13 in removing an implant from a patient in an operating room or elsewhere in which the user has fully covered the plurality of vents on an opposing side of the hollow container with the thumb of one hand; and

FIG. 17 is a perspective view of FIG. 13 of the use of the implant removal system of FIG. 13 after removal of the implant from a patient in an operating room in which the user has uncovered the plurality of vents with the index finger on one side of the hollow container.

### DETAILED DESCRIPTION

In the following detailed description, reference is made to the accompanying drawings that form a part hereof, and in which is shown, by way of illustration, specific embodiments in which the invention may be practiced. These embodiments are described in sufficient detail to enable those skilled in the art to practice the inventions, and it is understood that other embodiments may be utilized and that logical structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present inventions are defined only by the claims. Unless otherwise indicated, as used throughout this document, “or” does not require mutual exclusivity.

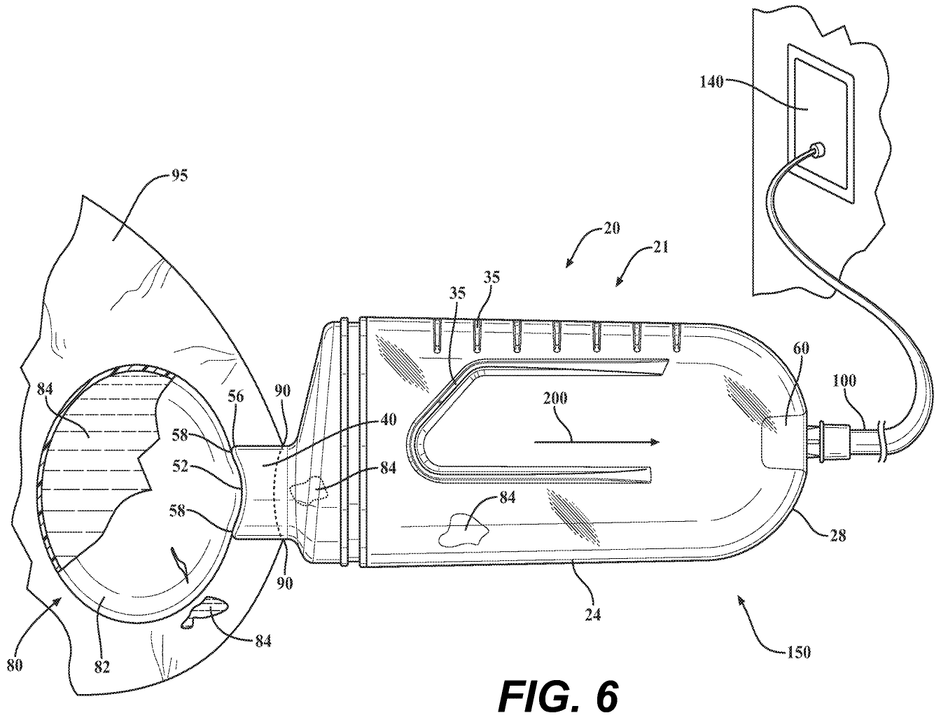

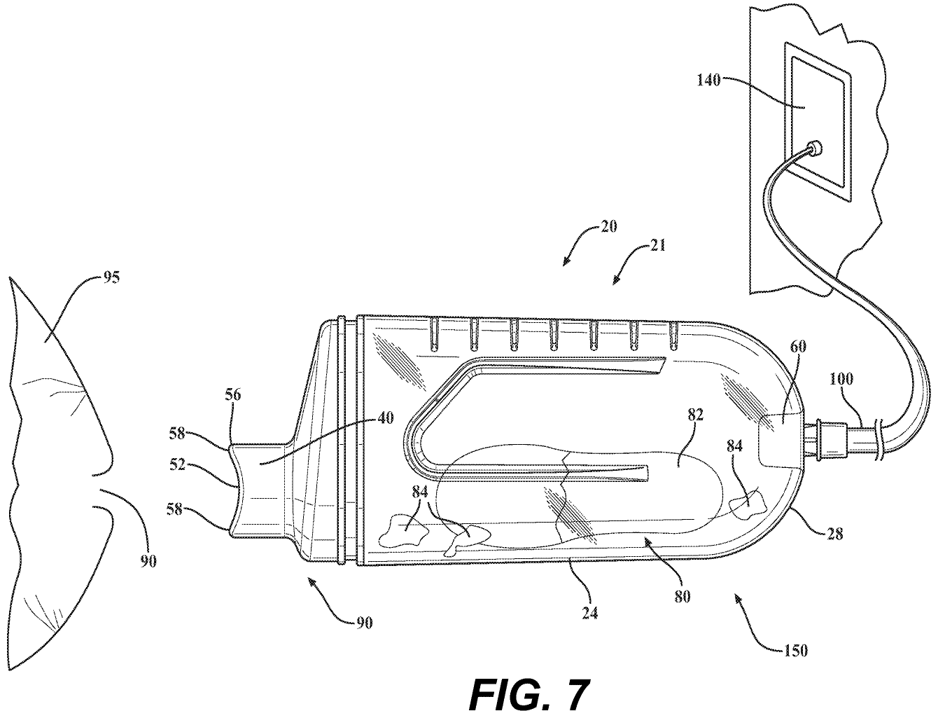

With reference to the Figures, an implant removal device 21 for use in an implant removal system 20 is disclosed herein that is configured for the removal of an implant, and in particular silicone gel-filled implants, from a patient during a surgical procedure. The disclosure also describes the use of the implant removal device 21 for removing an implant (shown as 80 in FIGS. 6 and 7 below), and in particular a silicone gel filled implant such as a breast implant, but could also be used in the removal of other implants that include a shell (shown as 82 in FIGS. 6 and 7 below) and a gel or other filling material (shown as silicone gel 84 in FIGS. 6 and 7 below), such as a saline-filled implant, a buttocks implant, or the like. For ease of description, the description of the shell 82 and the gel 84 as used hereinafter, refers to any type of implant 80 that includes such components, including but not limited to silicone-filled implants 80 such as illustrated in FIGS. 6 and 7.

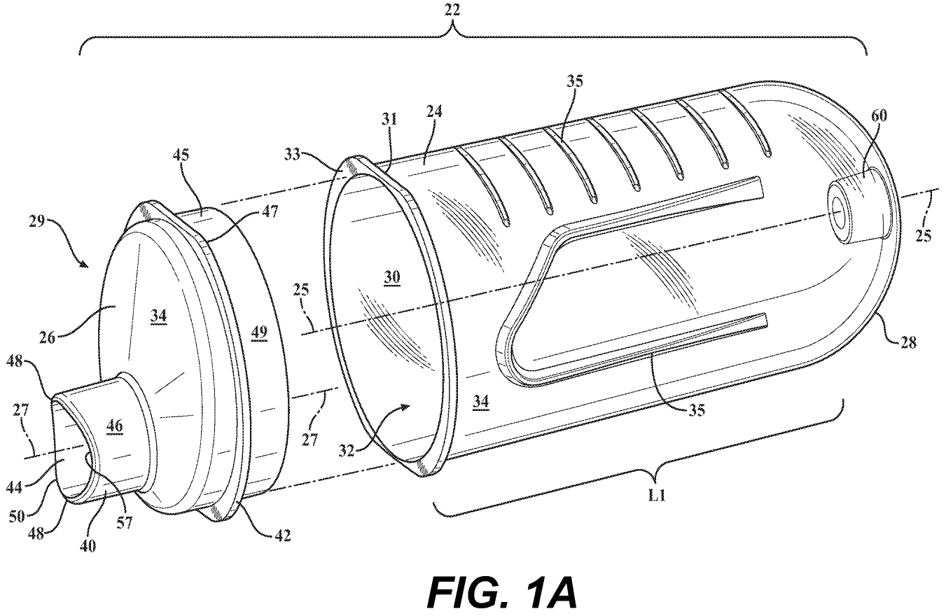

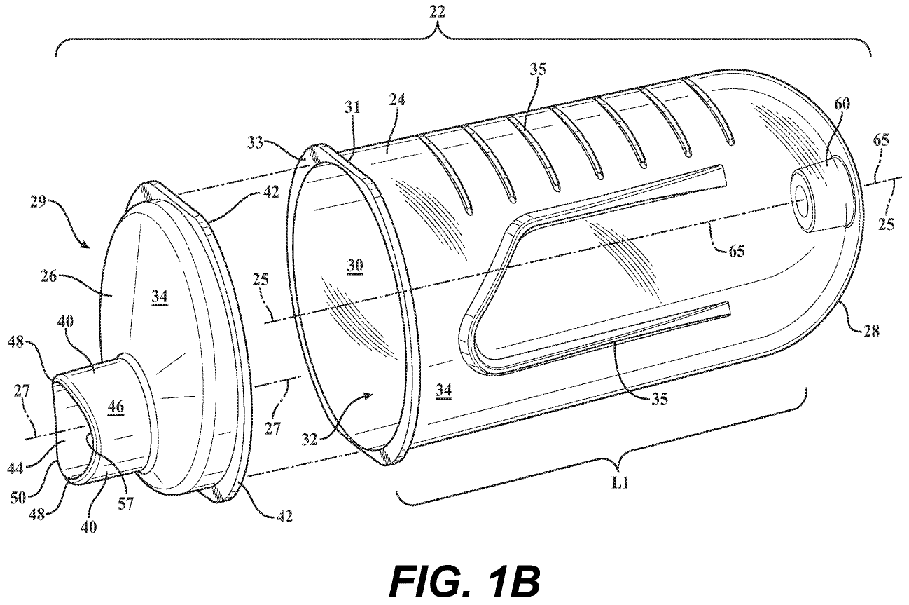

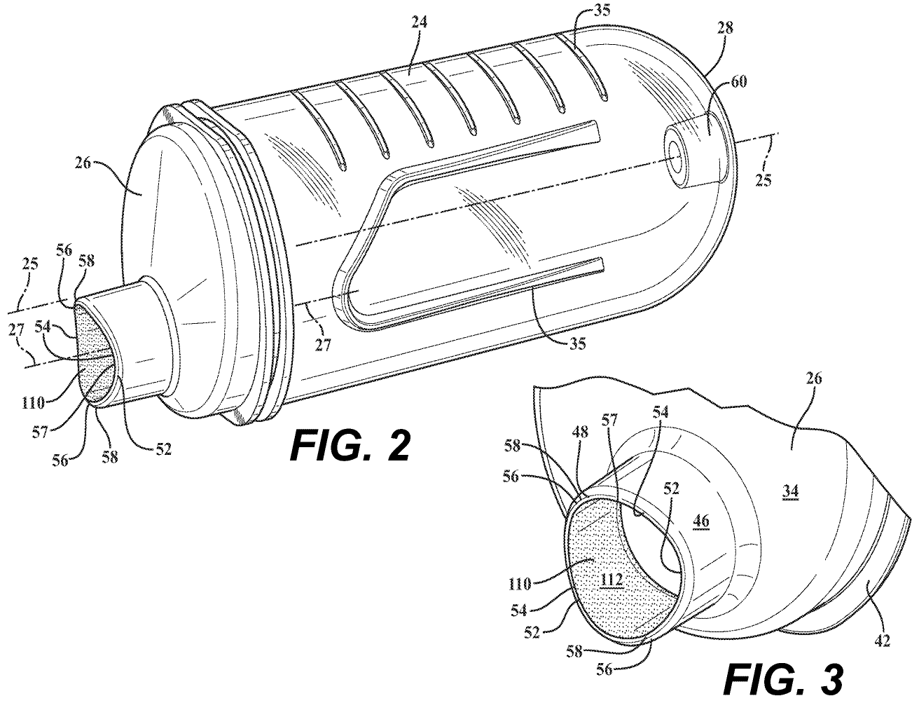

Referring first to FIGS. 1-5, the implant removal device 21 disclosed herein is in the form of a hollow container 22 having a middle portion 24 disposed between a first end 26 and a second end 28. In certain embodiments, the hollow container 22 is generally bottle-shaped, with the middle portion 24 being substantially circular or oval-shaped in cross-section. However, in other embodiments, the hollow container 22 may be shaped different. By way of example, as opposed to being substantially circular or oval-shaped in cross-section, the middle section 24 may be rectangular or triangular in cross-section, with the respective shapes of the first end 26 and second end 28 modified so as to correspond to the shape of the modified middle portion 24.

The middle portion 24, first end 26, and the second end 28 collectively have an interior container surface 30, which further defines a volume 32, and an exterior container surface 30 and the exterior container surface 34 defines a thickness of the hollow container 22 corresponding to the middle portion 24, first end 26, or the second end 28. The volume 32 of the hollow container 22 should be sufficient to contain the entirety of the implant shell and silicone gel of the implant (respectively identified by reference numerals 82, 84 and 80 in FIG. 6) removed during the surgical procedure. For example, in certain embodiments, the volume 32 may be between 100 and 5000 cubic centimeters, such as 1000 cubic centimeters, although in further embodiments smaller and larger volumes are contemplated.

In the embodiments illustrated, a longitudinal axis 25 may also be defined within the volume 32 that extends generally parallel to the length L1 of the middle portion 24 in a direction from the first end 26 to the second end 28. In embodiments wherein the middle portion 24 is symmetrical in cross-section, the longitudinal axis 25 may further defined as extending on a line that is equidistant from the interior container surface 30 of the middle portion 24.

To aid in gripping the exterior container surface 34 of the device 21, a series of one or more protrusions or recesses (shown here as protrusions 35) may be included on the exterior container surface 34 of the hollow container 22, such as on one or more protrusions 35 or recesses on the exterior container surface 34 of the middle portion 24, the first end 26, and/or the second end 28 (shown in FIGS. 1A, 1B, 2, 6 and 7) as being included on the middle portion 24).

A nozzle 40 is coupled to the first end 26 of the hollow container 22 which extends transversely in a direction away from the first end 26 and the second end 28. The nozzle 40 is configured for interfacing with the implant 80 through the surgical incision 90 (see FIG. 6) of a patient 95 during the surgical procedure to remove the implant 80. In certain embodiments, the positioning of the nozzle 40 is offset from the longitudinal axis 25, as will be described further below.

The nozzle 40 includes an interior nozzle surface 44 that extends outwardly from, or otherwise transitions outwardly from, the interior container surface 34 of the first end 26. In addition, the nozzle 40 includes an exterior nozzle surface 46 that extends outwardly from, or otherwise transitions outwardly away from, the exterior container surface 34 of the first end 26.

The interior container surface 44 of the nozzle 40 extends away from the first end 26 and terminates at a nozzle opening 50. The nozzle opening 50 functions as the inlet port for the receipt of the ruptured breast implant shell 82 and silicone gel 84 to the volume 32 of the hollow container 22 during the surgical procedure. The nozzle opening 50 is open to the volume 32 of the hollow container 22. The size of the nozzle opening 50 may vary depending upon the ultimate end use of the implant removal device 21. In particular, the size of the nozzle opening 50 may be enlarged or diminished in cases where larger amounts of material, including the ruptured breast implant 80, shell 82, or gel 84, is desired to be removed from the patient 95.

As noted above, in certain embodiments, the nozzle 40 is coupled to the first end 26 such that it is offset relative to the longitudinal axis 25. In particular, the nozzle 40 defines a nozzle axis 27 extending in a direction away from the first end 26, and preferably away and transverse to the first end 26, and in a direction that is parallel to, and radially spaced from, the longitudinal axis 25. With this configuration, the nozzle 40 is more easily positioned such that it may be located in closer proximity to the incision 90 along the skin of the patient 95 when the device 21 is used to remove the implant 80 during the implant removal process as compared with a nozzle 40 aligned along the longitudinal axis 25.

In certain embodiments, in addition to the nozzle opening 50 being offset from the longitudinal axis, the nozzle 40 is spaced from an exterior container surface 34 of the middle portion 24 toward the longitudinal axis 25.

In certain embodiments, the nozzle opening 50 includes at least one recessed portion 52 and at least one gripping portion 56. In further embodiments, the nozzle opening 50 includes a plurality of recessed portions 52 and a plurality of gripping portion 56, with each of the gripping portions 56 separated from each other by one of the respective pair of recessed portions 52, and vice versa. In the embodiments shown in FIGS. 1-7, the nozzle opening 50 includes a pair of recessed portions 52 and a pair of gripping portions 56, with each of the pair of gripping portions 56 separated from each other by one of the respective pair of recessed portions 52.

In certain embodiments, the gripping portions 56 define a terminal end surface 48 having an apex 58, with the apex 58 of each of the gripping portions 56 spaced further away from the first end 26 than the recessed portions 52.

Further, in certain embodiments, each of the recessed portions 52 are curved the gripping portions 56 to define a curved end surface 57. In these embodiments, the recessed portions 52 have a vertex 54 between respective apexes 58 of the gripping portions 56. The distance between the apex 58 of the gripping portions 56 and the vertex 54 of the recessed portions 52 define a depth D1 of the recessed portions 52.

When the implant removal device 20 is used to remove the implant 80, as will be described in further detail below with respect to the removal of a ruptured breast implant as illustrated in FIG. 6, the terminal end surface 48 corresponding to the apex 58 of each of the gripping portions 56 is brought into proximity with, and preferably brought into contact with, the shell 82 of the implant 80, and functions to aid in holding or otherwise gripping the shell 82 of the implant 80 during the removal process. In order to aid in this function, it is preferable that the nozzle 40, and in particular the gripping portions 56 of the nozzle opening 50, is formed from a material and has dimension to have sufficient rigidity that they resist deformation when contacting the shell 82 (i.e., the curved gripping portions 56 in particular and the nozzle 40 in general do not bend or collapse when contacting the shell 82), and in particular being pressed against the shell 82, during the process of removing the implant 80. Further, the nozzle 40 and nozzle opening 50 also resist deformation while contacting the shell 82 as described above while negative pressure is applied to the device 21 during the process of removing the implant 80.

It has been surprisingly and unexpectedly discovered that this offset positioning of the nozzle 40 relative to the longitudinal axis 25 and along the nozzle axis 27, in combination with the rigidity of the nozzle 40, and particularly in combination with the inclusion of one or more gripping portions 56 on the nozzle 40, provides a maximum efficiency in the removal of the implant shell 82 and silicone gel 84 of the implant 80 during the removal process, as will be described in further detail below.

In certain embodiments, such as shown in the Figures herein, the first end 26 and the nozzle 40 are a one-piece structure, with the one-piece structure being is coupled to, or otherwise secured to, the distinct middle portion 24 to form the hollow container 22 prior to use.

Stated another way, the nozzle 40 is integrally formed with the first end 26 as a one-piece structure which is distinct from the structure of the middle portion 24 and second end 28.

In certain embodiments, the hollow container 22 includes a first cap 29 defining the first end 26, with an outer peripheral end 42 of the first cap 29 being coupled to the middle portion 24. In particular, the outer peripheral end 42 of the first cap 29 is positioned adjacent to, or is otherwise coupled or sealed to, a terminal end region 31 of the middle portion 24. In embodiments described below, the first cap 29 is shown as being press fit and/or ultrasonically welded to the middle portion 24.

In certain of these embodiments, as best shown in FIGS. 1A, 4, and 5, the outer peripheral end 42 of the first cap 29 includes a ledge 45 extending transverse from a shoulder 47. The ledge 45 and shoulder 47 are shaped such that the outer surface 49 of the ledge 45 is press fit against the interior container surface 30 of the terminal end region 31 of the middle portion 24 and wherein the inward surface 51 of the shoulder 47 is adjacent to the outer terminal surface 33 of the middle portion 24.

In certain alternative embodiments (not shown), as opposed to press fitting the ledge 45 of the first cap 29 to the interior container surface 30 of the middle portion 24, the exterior container surface 34 of the middle portion 24 may be press fit to an inner surface 51 of the ledge 45 of the first cap 29, with the inward surface 51 of the shoulder 47 still being adjacent to the outer terminal surface 33 (but wherein the shoulder 47 extends inwardly instead of outwardly. In certain related embodiments, in order to permanently secure the first cap 29 to the middle portion 24 in these alternative embodiments, an ultrasonic welding process may be performed, or an adhesive may be included, between the outer surface 49 and interior container surface 30 (or in the alternative arrangement between the inner surface 51 and the exterior container surface 34), and/or between the inward surface 51 and the outer terminal surface 33 to secure or otherwise affix the first cap 29 to the middle portion 24 if desired.

In another alternative embodiment of the first cap 29 as shown in FIG. 1B, as opposed to a press fitting the outer peripheral end 42 of the first cap 29 to the middle portion 24, an inward terminal surface 55 of the outer peripheral end 42 of the first cap 29 may simply be abutted to the outer terminal surface 33 of the outer terminal region 31 of the middle portion 24 and be secured thereto via ultrasonic welding or through the use of an adhesive.

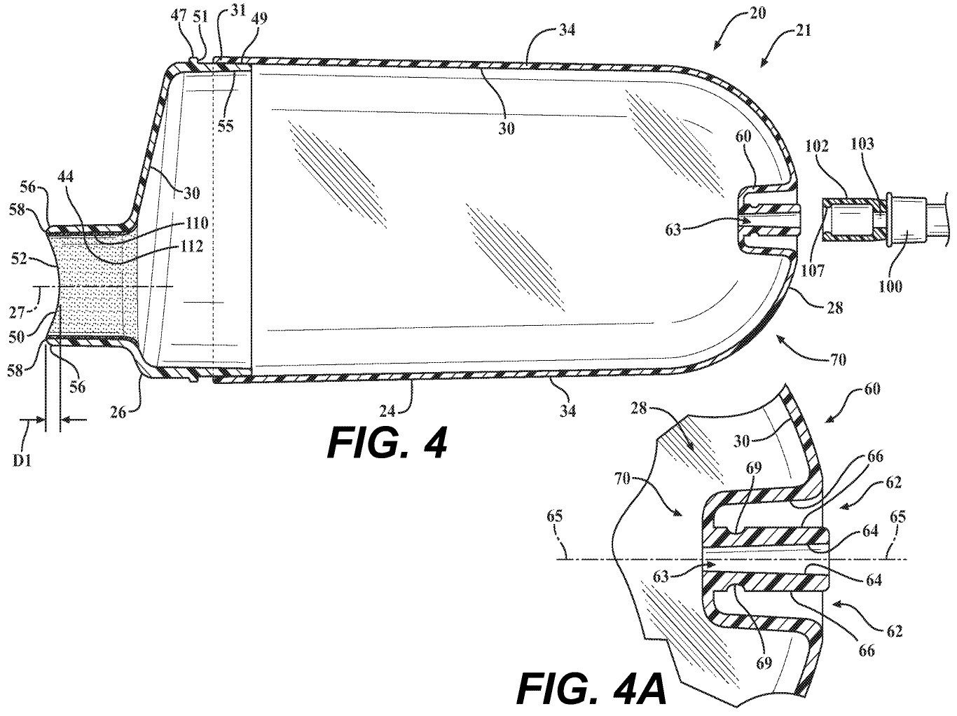

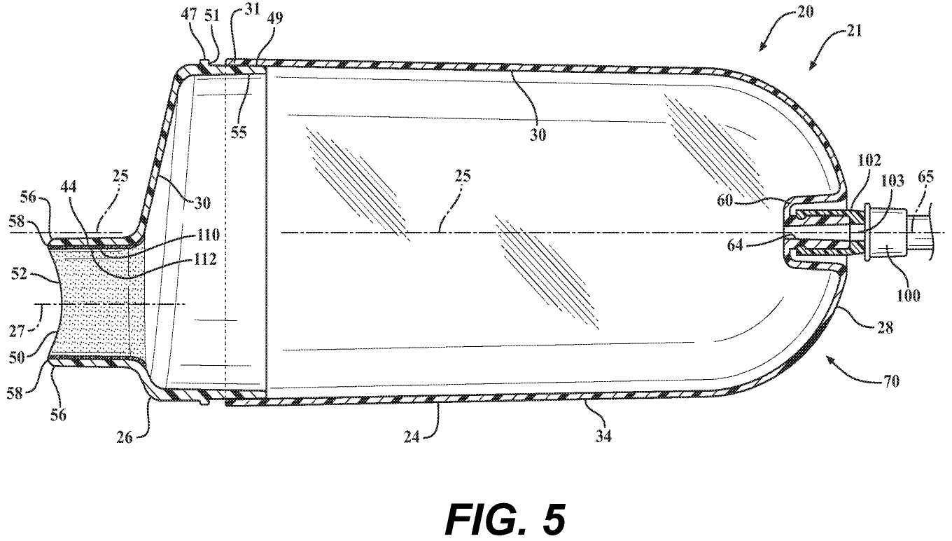

In certain embodiments, a connector port 60 is coupled to the second end 28 of the hollow container 22. The connector port 60, as best shown in FIG. 4A, includes a connector receiving region 62 that is adapted for connection to the suction device 100. In particular, as further shown in FIG. 5, the connector interior region 62 is adapted to receive an external portion 102 of the suction device 100.

In certain embodiments, the connector interior region 62 has an internal opening 63 that is open to, and forms a portion of, the volume 32 of the hollow container 22. The internal opening 63 of the connector interior region 62 is also open with an internal opening 103 of the suction device 100 when the suction device 100 is coupled to the connector port 60, with the internal opening 103 defined within the external portion 102. As such, when negative pressure is actuated from the suction device 100, suction is created (shown as arrow 200 in FIG. 6) through the internal opening 103, the internal opening 63, and into the volume 32 of the hollow container 22 that is then used that draw the implant 80 (including the shell 82 and gel 84) through the nozzle opening 50 and into the volume 32.

In certain embodiments, such as shown in FIGS. 4 and 4A, the connector port 60 is integrally formed with the second end 28. In these embodiments, the inner surface 64 of the connector receiving region 62, which defines the internal opening 63, extends outwardly from, or otherwise transitions outwardly from the interior container surface 30 of the second end 28 in an opposite direction from the middle portion 24 relative to the nozzle 40. Similarly, the connector receiving region 62 includes an outer surface 66 that extends outwardly from, or otherwise transitions outwardly away from the exterior container surface 34 of the first end 26. The outer surface 66 also includes a depressed region 69 that receives a corresponding flange 107 of the external portion 102 of the suction device 100 to secure the suction device 100 to the connector portion 60.

The connector port 60 also defines a connector axis 65. In particular, the connector axis 65 is defined within the internal opening 63 and is generally aligned parallel to the length of the inner surface 64 of the connector port 60.

In certain embodiments, such as shown in the Figures, the connector axis 65 is parallel to the nozzle axis 27. In further embodiments, the connector axis 65 is offset from the longitudinal axis 25. Still further, in certain embodiments, the connector axis 65 is offset from the longitudinal axis 25 and is radially spaced from the nozzle axis 27 relative to the longitudinal axis 25.

In still further embodiments, the connector port 60 is coupled to the second end 28 and extends in a direction transverse to the longitudinal axis 25. In certain of these embodiments, the connector port 60 is coupled to the second end 28 and extends in a direction normal to the longitudinal axis 25.

In still further embodiments, as opposed to being coupled to the second end 28, the connector port 60 is coupled to the middle portion 24 and extends in a direction transverse to the longitudinal axis 25. In certain of these embodiments, the connector port 60 is coupled to the middle portion 24 and extends in a direction normal to the longitudinal axis 25.

In certain other embodiments, the connector port 60 is a separate structure that is coupled to the second end 28. In certain of these embodiments, the outer surface 66 of the connector port 60 is coupled to the interior container surface 30 of the second end 28, while in further alternative embodiments the inner surface 64 of the connector port 60 is coupled around the exterior container surface 34 of the second end 28. In either of these embodiments, ultrasonic welding or an adhesive material may be introduced between the connector port 60 and the second end 28 to secure the connector port 60 to the first end 28. In embodiments wherein the connector port 60 is coupled to the middle portion 24 and is a separate structure from the middle portion 24, a similar kind of coupling and securing via ultrasonic welding or through an adhesive can occur.

In certain embodiments, the hollow container includes a second cap 70 defining the second end 28. In these embodiments, the second cap 70 is integrally formed with the middle portion 24 as a one-piece structure, such s best shown in FIGS. 4 and 5.

Alternatively, the second cap 70 may be coupled to another terminal end surface (not shown) of the middle portion 24 in a manner like the connection of the first cap 29 to the terminal end region 31 of the middle portion 24 as described above.

The hollow container 22, in certain embodiments, is formed from a material having physical properties, particularly in terms of leakage prevention, durability and strength, appropriate for the temporary acceptance of the shell 82, any silicone gel 84, and associated medical waste from the patient 95. Preferably, the hollow container 22 is transparent such that a doctor can confirm the acceptance of the breast implant shell 82, any silicone gel 84, and associated medical waste from the patient 95 during the surgical procedure. In certain embodiments, however, the separate first end 26 (including the nozzle 40) may be formed from an opaque material, such as an opaque plastic material. Still further, as noted above, the material used in the nozzle 40 should have sufficient rigidity to maintain its shape when pressed against the shell 82 of the implant 80 during the removal process. Yet still further, it is desirable that the material used in forming the hollow container 22 has sufficient rigidity and strength to withstand collapsing upon the introduction of negative pressure, in the range of 150-500 mm Hg (mercury), during the removal process. Even still further, it is desirable that such material retain their physical properties, including transparency, after a sterilization process such as through e-beam sterilization.

Illustrative materials for forming the hollow container 22, or any component thereof (such as the nozzle 40), include, but are not limited, thermoplastic materials such as polycarbonate. In certain embodiments, the materials for each separate piece of the hollow container 22 are formed from the same illustrative material, while in other embodiments the separate pieces may be formed of different materials, or slightly different formulations of the same general thermoplastic material. For example, in embodiments, wherein the first end 26 is formed from an opaque material, while the middle portion 24 and second end 28 are formed from a transparent material, the difference in such materials could simply occur by adjusting the formulations to include additives (such as including a fillers or pigments in the first end 26 that are not present, or present in differing amounts, in the middle portion 24 or second end 28). Alternatively, the polymer composition of the thermoplastic materials may be different (such as through the use of a polycarbonate material in one instance and a polyurethane in another instance, or alternatively wherein the generally polycarbonate material is modified in some other manner (such as through changes in number average molecular weights or by modifying the structure to include additional chemical groups). Other materials, such as thermosetting polymeric materials or glass, may also form one or more of the separate components of the hollow container 22.

In certain embodiments, a coating layer 110 is applied to the interior nozzle surface 44 of the nozzle 40. The coating layer 110 is in the form of a low friction coating layer 110 formed from a low friction material, in which the outer surface 112 (shown in FIG. 5) of the coating layer 110, opposite the interior nozzle surface 44, has lower surface friction than the corresponding surface friction of the interior nozzle surface 44. In certain embodiments, in addition to forming a low surface friction outer surface, the coating material also is hydrophilic coating, which is believed to provide the outer surface layer with an increased amount of lubricity.

The lower surface friction properties of the outer surface 112 of the coating layer 110, alone or in combination with the increase in hydrophilicity, aids in preventing the implant 80 and any associated residual medical waste (such as blood, tissue, water etc.) from a patient from adhering onto the outer surface 112 during use of implant removal device 21 in removing the implant 80 as compared with an uncoated interior nozzle surface 44, thereby increasing the efficiency of the implant removal process.

In still further embodiments, in addition to having the lower surface friction properties, the outer surface 112 of the coating layer 110 may also provide increased durability properties, or enhanced durability properties, as compared with devices 21 not include such a coating layer 110. Further, the coating layers 110 provided have sufficient bonding strength to the interior nozzle surface 44 such that they are not removed, delaminated, or otherwise degrade during the implant removal process.

Illustrative, non-limiting coating compositions used to form the coating layer 110 include low friction, hydrophilic coating compositions sold under the tradename Serene™, commercially available from Surmodics, Inc. of Eden Prairie, Minnesota and hydrophobic coating compositions sold under the tradename Hydak®, commercially available from Biocoat, Inc., of Horsham, Pennsylvania. However, other coating compositions for forming layers 110 having specific properties or combination of properties are also contemplated.

In even further embodiments, a flexible diaphragm may be provided that is positioned at the interface between the hollow container 22 and the suction device 100, or within the interior of the hollow container 22 at a position near, along or adjacent to the connector port 60, to separate the volume 32 of the hollow container 25 from the suction device 100. The flexible diaphragm functions to limit contamination of the suction device 100 from the removed implant 80 during use, the flexible diaphragm also prevents the introduction of cross contamination into the volume 32 of the hollow container 22, and into the patient 95 via the volume 32 of the hollow container 22, from the outside environment. When included, the flexible diaphragm does not adversely impact the generation of negative pressure from the suction device 100 within the volume 32 of the hollow container 22.

The present invention also is directed to an implant removal system 20, including the afore-mentioned implant removal device 21, and an associated method which is used in a surgical procedure to remove a ruptured silicone breast implant 80 from a patient 95. In the implant removal system 20, as shown in FIG. 6, the implant removal device 21 is coupled to a suction device 100, such as a wall suction device 100 coupled to and extending from a wall 140 within an operating room 150 which when actuated provides negative pressure, in the range of 150-500 mm Hg, within the volume 32 of the hollow container 22 and through the nozzle opening 50 sufficient to facilitate the removal of the shell 82 and silicone gel 84 from the patient 95.

The suction device 100 is conventional in nature and is of the type found in hospital or medical settings to provide a vacuum source for surgical procedures. The suction device 100 may be mounted in an operating room 150 of a medical facility, or alternatively may be mobile.

In particular, as best shown in FIG. 6, during the surgical procedure for removal on an implant, here shown as ruptured silicone breast implant 80, a small incision 90 is made in the skin of a patient 95 in order to access the ruptured implant 80.

The device 21, which has been sterilized before use such as through e-beam sterilization as described above, is then brought into proximity with the incision 90 and is utilized to remove the implant 80. More particularly, the device 21 is positioned on the surface of the skin near the incision 90 between the ends of the incision 90, with the nozzle opening 50 positioned in close proximity to the skin of the patient 95 for easier insertion. The nozzle 40 is then inserted within the small incision 90 in the skin and brought into proximity to the implant 80. The size of the small incision 90 is preferably roughly equal to the cross-sectional width to the nozzle 40 such that the nozzle 40 is surrounded bye the tissue and skin of the patient 95 after insertion, although the size could be larger if desired based on the preference of the doctor.

Preferably, during the insertion process, the gripping portions 156 are brought into pressing contact with the shell 82 of the implant 80. As noted above, the rigidity of the nozzle 40 and the gripping portions 156 allows the device 21 to maintain its internal position within the incision 90 of the patient 20 during the implant removal process.

The suction device 100 is coupled to the connector port 60 either prior to the insertion of the nozzle 40 or after the insertion of the nozzle 40. Typically this is accomplished by inserting the external portion 102 of the suction device 100 into the connector receiving portion 62 and securing the flange 107 into the depressed portion 107 of the outer surface 66. The suction device 100 is then actuated, thereby creating negative pressure (i.e., vacuum suction) within the volume 32 of the hollow container 22 (shown by arrow 200 in FIG. 6) sufficient to draw out the breast implant shell 82 and cohesive silicone gel 84 through the nozzle opening 50 of the device 21 and into the volume 32 of the hollow container 22. As illustrated in FIG. 6, a portion of the silicone gel 84 has been removed to the volume 32, while the majority of the gel 84 contained in the shell 82 still remains in the patient 95, such as immediately after actuation. As noted above, in certain embodiments, such negative pressure may be between 150 and 500 mm Hg. If needed, the negative pressure of the suction device 100 may be increased or decreased relative to the desired range. Because the middle portion 24 of the device 21 is transparent, the doctor can observe and confirm the introduction of the shell 82 and silicone gel 84 in the volume 32 of the hollow container 22 during the removal process as the negative pressure is actuated.

Upon confirmation, typically visual confirmation by the doctor, that a desired amount of the implant shell 82 and silicone gel 84 has been removed from the patient 95 and is visible through the transparent middle portion 24 in the volume 32, the suction device 100 is turned off, and the device 21 is removed from the incision 90, as shown in FIG. 7. The suction device 100 is then disconnected from the connector port 60. Any residual silicone gel 84 or shell 82 of the implant 80 that was not removed by the device 21 can be removed from the patient through the use of surgical sponges or other known manual extraction surgical techniques.

The device 21, including the extravagated silicone gel 84 and shell 82 contained within the volume 32 of the hollow container 22 as best illustrated in FIG. 7, may be disposed of as medical waste. Alternatively, such materials may be removed from the device 21, and the device 21 can be cleaned, sterilized, and reused as desired.

The disclosed devices differ from what currently exists. For example, there is no implant removal device 21 available which is able to be sterilized prior to use and will assist in removal of an implant 80 (or the shell or gel), such as a ruptured breast implant, as is described herein. This invention is an improvement on what currently exists. The device removes the vast majority of the extravagated silicone and the shell and traps it into an easily disposed unit which substantially expedite the process as well as limits contamination.

While the device 21 is ideally suited for the removal of silicone breast implants, and in particular ruptured silicone breast implants, the device 21 is also appropriate for use, in the method described above, for removal of other types of implants having a shell and material contained within a shell (such as silicone gel, saline, or some other filling material).

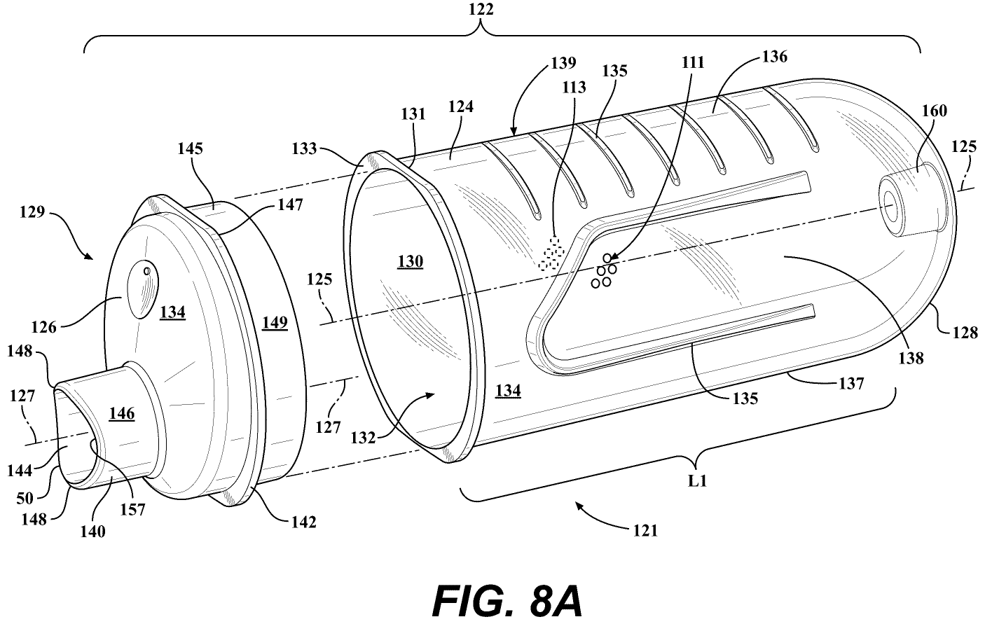

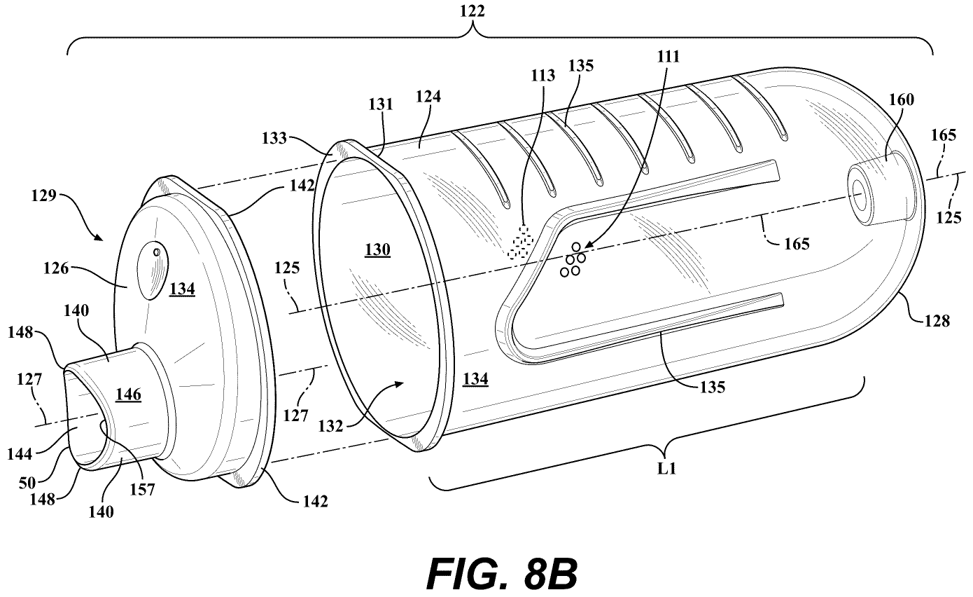

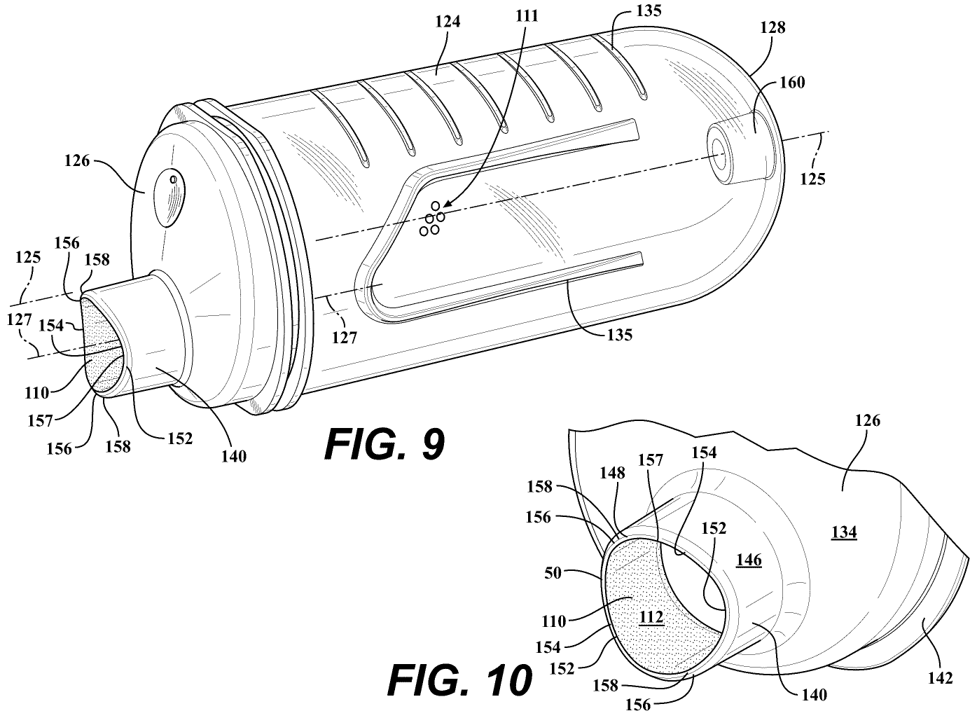

Referring now to FIGS. 8A-17, an illustrative embodiment of an implant removal device 121 for use in an implant removal system 120 will be described in more detail. The implant removal device 121 and the implant removal system 120 are similar to the implant removal device 21 and the implant removal system 20 described above with reference to FIGS. 1A-7. However, the implant removal device 121 and implant removal system 120 include additional features that will be described in detail below.

The implant removal device 121 and implant removal system 120 are configured for the removal of the same types of implants from the same type of surgical sites as those described above with reference to FIGS. 1A-7. Thus, the same reference numerals for the implant 80, the shell 82, the gel 84, the surgical incision 90 and the patient 95 are used with reference to FIGS. 8A-17.

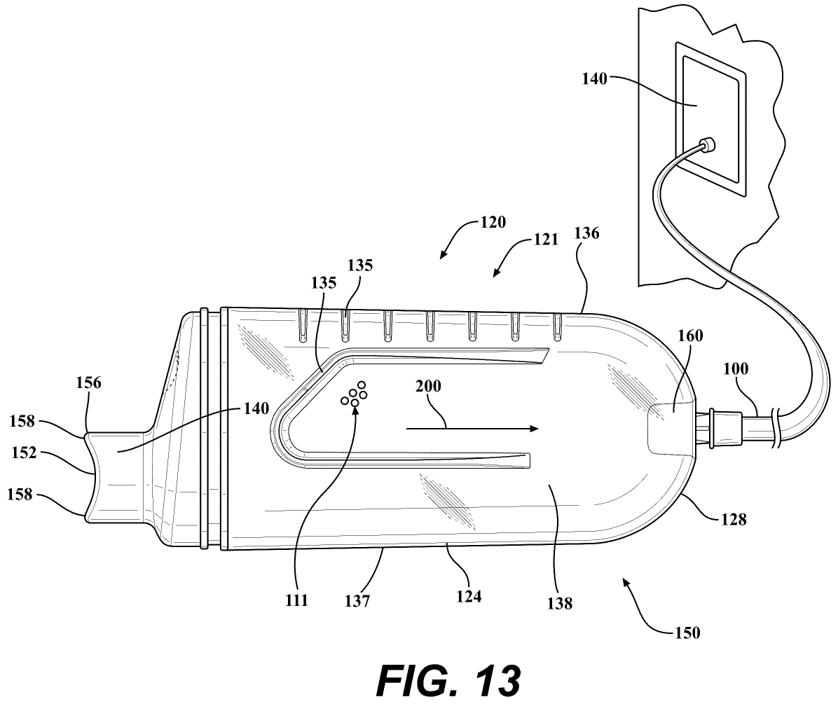

Referring primarily to FIGS. 8A-12, with occasional reference to FIGS. 13-17, the implant removal device 121 includes a hollow container 122 having a middle portion 124 disposed between a first end 126 and a second end 128. In certain embodiments, the hollow container 122 is generally bottle-shaped, with the middle portion 124 being substantially circular or oval-shaped in cross-section. However, in other embodiments, the hollow container 122 may have other shapes like those described above with reference to the implant removal device 21 of FIGS. 1A-7.

The middle portion 124, the first end 126, and the second end 128 collectively have an interior container surface 130, which further defines a volume 132, and an exterior container surface 134 opposite the interior container surface 130. The distance between the interior container surface 130 and the exterior container surface 134 defines a thickness of the hollow container 122. The volume 132 of the hollow container 122 should be sufficient to contain the entirety of the implant shell 82 and the silicone gel 84 of the implant 80 removed during the surgical procedure. For example, in certain embodiments, the volume 132 may be between 100 and 5000 cubic centimeters. In some embodiments the volume 132 is 1000 cubic centimeters. In yet other embodiments, smaller and larger volumes are contemplated should a less common size implant be removed.

In the embodiments illustrated, a longitudinal axis 125 may also be defined within the volume 132 that extends generally parallel to the length L1 of the middle portion 124 in a direction from the first end 126 to the second end 128. In embodiments wherein the middle portion 124 is symmetrical in cross-section, the longitudinal axis 125 may be further defined as extending on a line that is equidistant within the interior container surface 130 of the middle portion 124.

To aid in gripping the exterior container surface 134 of the device 121, a series of one or more protrusions or recesses (shown here as protrusions 135) may be included on the exterior container surface 134 of the hollow container 122. The protrusions 135 may be located on the middle portion 124, the first end 126, and/or the second end 128 of the exterior container surface 134.

Referring now to FIGS. 8A-17, the middle portion 124 of the hollow container 122 can be further defined as including a top side 136, a bottom side 137, a first side 138, and a second side 139 (which is opposite the first side 138), with the first side 138 and the second side 139 each connecting the top side 136 to the bottom side 137. In one embodiment, a first set of vent holes 111 is formed in the first side 138, such that the first set of vent holes 111 extend between the interior container surface 130 and the exterior container surface 134. In another embodiment, a second set of vent holes 113 is formed in the second side 139, such that the second set of vent holes 113 extend between the interior surface container 130 and the exterior surface container 134. In yet another embodiment, the first and second sides 138, 139 have their respective set of vent holes 111, 113 formed therethrough. The open vent holes allow gas to pas therethrough between the exterior and interior. It should be appreciated that a set of vent holes may include one or more vent holes. In some aspects, the set of vent holes may include 1, 2, 3, 4, 5, 6, or more holes. As will be described below, the first and second set of vent holes 111 or 113 are configured to reduce negative pressure within the hollow container 122 when the suction device 100 is activated during a removal procedure (see FIGS. 13-17). Negative pressure may be used to refer to reduced pressure from ambient.

In the embodiments illustrated, the first set of vent holes 111 (see FIGS. 8A, 8B, 9, and 13-17) is positioned on the first side 138 of the middle portion 124 of the hollow container 122, while the second set of vent holes 113 (see FIGS. 8A, 8B, 11, 12, and 16) is positioned on the second side 139 of the middle portion 124 of the hollow container 122.

The number, size, shape, and positioning of the first set of vent holes 111 on the first side 138 of the middle portion 124 of the hollow container 122 may vary, but preferably are configured in such a manner that each of the first set of vent holes 111 may be fully covered, partially covered, or uncovered by one or more fingers (collectively and individually labelled as reference numeral 205) on one hand 200 of an operator, and more preferably by one finger 205 such as the index finger 210 on the one hand 200 of the operator, when utilizing the device 121 during a removal process.

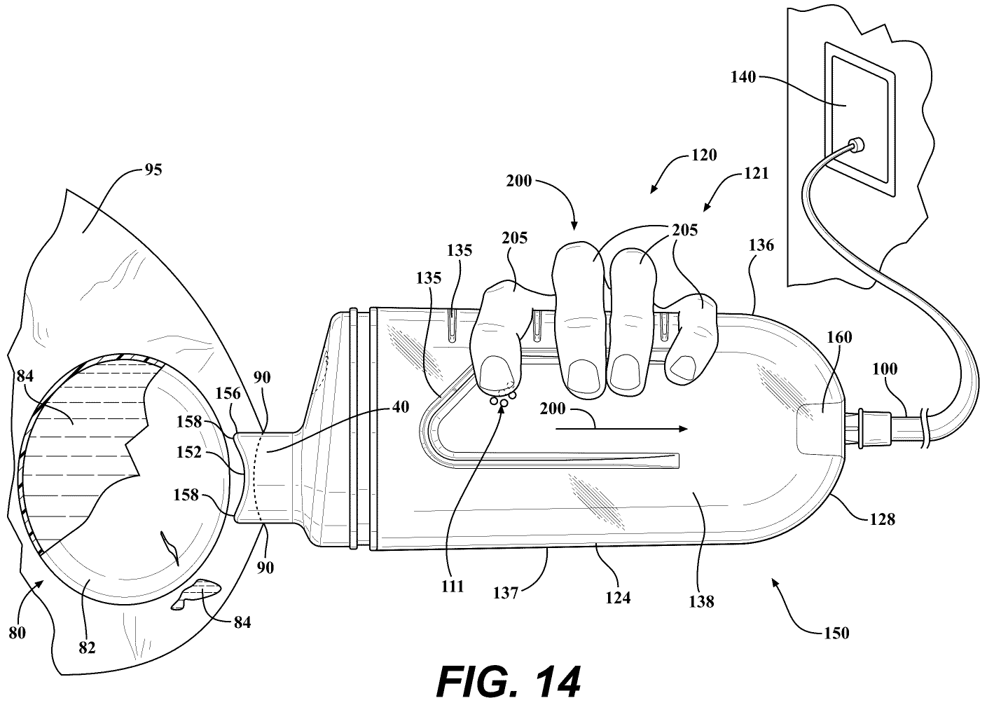

Similarly, the number, size, shape, and positioning of the second set of vent holes 113 on the second side 139 of the middle portion 124 of the hollow container 122 may vary, but preferably are configured in such a manner that each of the first set of vent holes 113 may be fully covered, partially covered, or uncovered by a thumb 220 on the one hand 200 of an operator when utilizing the device 121 during a removal process. As intended herein, the fingers 205 and thumb 220 are on the same hand (i.e., either the right hand or the left hand, shown as the right hand 200 in FIGS. 14-17).

A nozzle 140 is coupled to the first end 126 of the hollow container 122 which extends transversely in a direction away from the first end 126 and the second end 128. The nozzle 140 is configured for interfacing with the implant 80 through the surgical incision 90 (see FIG. 14) of a patient 95 during the surgical procedure to remove the implant 80. In certain embodiments, the positioning of the nozzle 140 is offset from the longitudinal axis 125, as will be described further below.

The nozzle 140 includes an interior nozzle surface 144 that extends outwardly from, or otherwise transitions outwardly from, the interior container surface 130 of the first end 126. In addition, the nozzle 140 includes an exterior nozzle surface 146 that extends outwardly from, or otherwise transitions outwardly away from, the exterior container surface 134 of the first end 126.

The interior container surface 144 of the nozzle 140 extends away from the first end 126 and terminates at a nozzle opening 50. The nozzle opening 50 functions as the inlet port for the receipt of the ruptured breast implant shell 82 and silicone gel 84 to the volume 132 of the hollow container 122 during the surgical procedure. The nozzle opening 50 is open to the volume 132 of the hollow container 122. The size of the nozzle opening 50 may vary depending upon the ultimate end use of the implant removal device 121. In particular, the size of the nozzle opening 50 may be enlarged or diminished in cases where larger amounts of material, including the ruptured breast implant 80, shell 82, or gel 84, is desired to be removed from the patient 95.

As noted above, in certain embodiments, the nozzle 140 is coupled to the first end 126 such that it is offset relative to the longitudinal axis 125. In particular, the nozzle 140 defines a nozzle axis 127 extending in a direction away from the first end 126, and preferably away and transverse to the first end 126, and in a direction that is parallel to, and radially spaced from, the longitudinal axis 125. With this configuration, the nozzle 140 is more easily positioned such that it may be located in closer proximity to the incision 90 along the skin of the patient 95 when the device 121 is used to remove the implant 80 during the implant removal process as compared with a nozzle 140 aligned along the longitudinal axis 125.

In certain embodiments, in addition to the nozzle opening 50 being offset from the longitudinal axis, the nozzle 140 is spaced from an exterior container surface 134 of the middle portion 124 toward the longitudinal axis 125.

In certain embodiments, the nozzle opening 50 includes at least one recessed portion 152 and at least one gripping portion 156. In further embodiments, the nozzle opening 50 includes a plurality of recessed portions 152 and a plurality of gripping portion 156, with each of the gripping portions 156 separated from each other by one of the respective pair of recessed portions 152, and vice versa. In the embodiments shown in the Figures, the nozzle opening 50 includes a pair of recessed portions 152 and a pair of gripping portions 156, with each of the pair of gripping portions 156 separated from each other by one of the respective pair of recessed portions 152.

In certain embodiments, the gripping portions 156 define a terminal end surface 148 having an apex 158, with the apex 158 of each of the gripping portions 156 spaced further away from the first end 126 than the recessed portions 152.

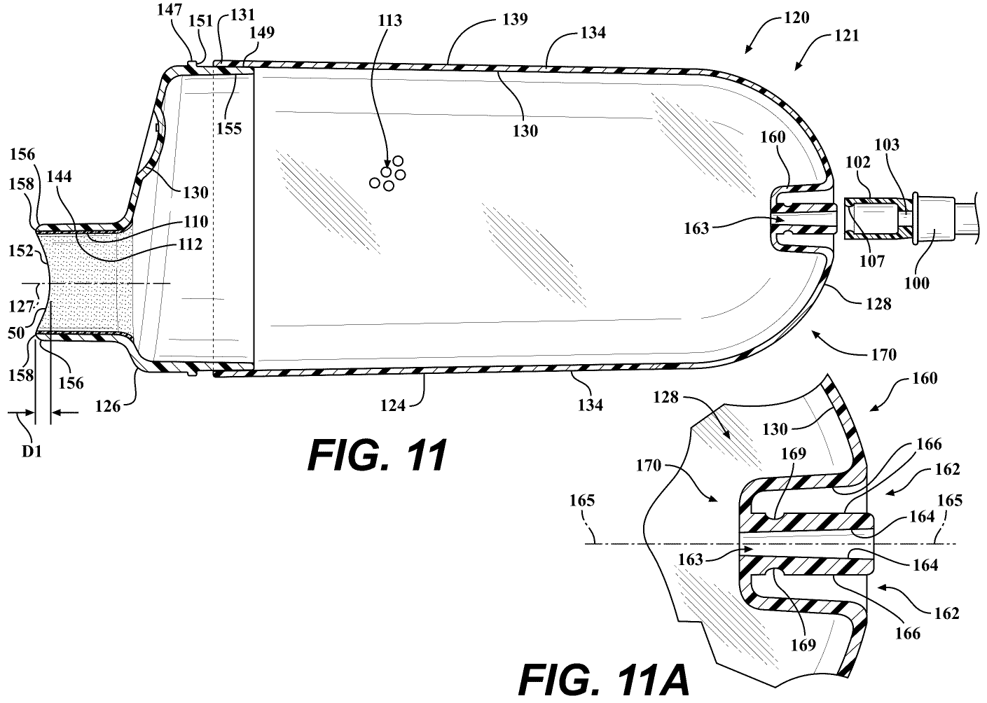

Further, in certain embodiments, each of the recessed portions 152 are curved so as to define a curved end surface 157. In these embodiments, the recessed portions 152 have a vertex 154 between a respective apexes 158 of the gripping portions 156. The distance between the apex 158 of the gripping portions 156 and the vertex 154 of the recessed portions 152 define a depth D1 (see FIG. 11) of the recessed portions 152.

When the implant removal device 120 is used to remove the implant 80, as will be described in further detail below with respect to the removal of a ruptured breast implant as illustrated in FIGS. 14-17, the terminal end surface 148 corresponding to the apex 158 of each of the gripping portions 156 is brought into proximity with, and preferably brought into contact with, the shell 82 of the implant 80, and functions to aid in holding or otherwise gripping the shell 82 of the implant 80 during the removal process. In order to aid in this function, it is preferable that the nozzle 140, and in particular the gripping portions 156 of the nozzle opening 50, is formed from a material and has dimension to have sufficient rigidity that they resist deformation when contacting the shell 82 (i.e., the curved gripping portions 156 in particular and the nozzle 140 in general do not bend or collapse when contacting the shell 82), and in particular being pressed against the shell 82, during the process of removing the implant 80. Further, the nozzle 140 and nozzle opening 50 also resist deformation while contacting the shell 82 as described above while negative pressure is applied to the device 121 during the process of removing the implant 80.

It has been surprisingly and unexpectedly discovered that this offset positioning of the nozzle 140 relative to the longitudinal axis 125 and along the nozzle axis 127, in combination with the rigidity of the nozzle 140, and particularly in combination with the inclusion of one or more gripping portions 156 on the nozzle 140, provides a maximum efficiency in the removal of the implant shell 82 and silicone gel 84 of the implant 80 during the removal process, as will be described in further detail below.

In certain embodiments, such as shown in the Figures herein, the first end 126 and the nozzle 140 are a one-piece structure, with the one-piece structure being coupled to, or otherwise secured to, the distinct middle portion 124 to form the hollow container 122 prior to use. Stated another way, the nozzle 140 is integrally formed with the first end 126 as a one-piece structure which is distinct from the structure of the middle portion 124 and second end 128.

In certain embodiments, the hollow container 122 includes a first cap 129 defining the first end 126, with an outer peripheral end 142 of the first cap 129 being coupled to the middle portion 124. In particular, the outer peripheral end 142 of the first cap 129 is positioned adjacent to, or is otherwise coupled or sealed to, a terminal end region 131 of the middle portion 124. In embodiments described below, the first cap 129 is shown as being press fit and/or ultrasonically welded to the middle portion 124.

In certain of these embodiments, as best shown in FIGS. 8A, 11 and 12, the outer peripheral end 142 of the first cap 129 includes a ledge 145 extending transverse from a shoulder 147. The ledge 145 and shoulder 147 are shaped such that the outer surface 149 of the ledge 145 is press fit against the interior container surface 130 of the terminal end region 131 of the middle portion 124 and wherein the inward surface 151 of the shoulder 147 is adjacent to the outer terminal surface 133 of the middle portion 124.

In certain alternative embodiments, as opposed to press fitting the ledge 145 of the first cap 129 to the interior container surface 130 of the middle portion 124, the exterior container surface 134 of the middle portion 124 may be press fit to an inner surface 151 of the ledge 145 of the first cap 129, with the inward surface 151 of the shoulder 147 still being adjacent to the outer terminal surface 133 but wherein the shoulder 147 extends inwardly instead of outwardly. In certain related embodiments, in order to permanently secure the first cap 129 to the middle portion 124, an ultrasonic welding process may be performed, or an adhesive may be included, between the outer surface 149 and interior container surface 130 (or in the alternative arrangement between the inner surface 151 and the exterior container surface 134), and/or between the inward surface 151 and the outer terminal surface 133 to secure or otherwise affix the first cap 129 to the middle portion 124 if desired.

In another alternative embodiment of the first cap 129 as shown in FIG. 8B, as opposed to a press fitting the outer peripheral end 142 of the first cap 129 to the middle portion 124, an inward terminal surface 155 of the outer peripheral end 142 of the first cap 129 may simply be abutted to the outer terminal surface 133 of the outer terminal region 131 of the middle portion 124 and be secured thereto via ultrasonic welding or through the use of an adhesive.

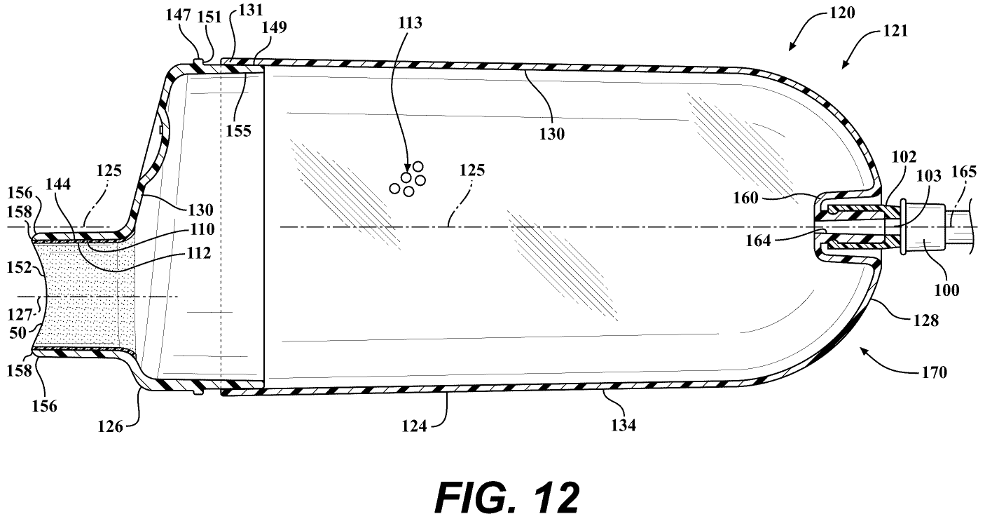

In certain embodiments, a connector port 160 is coupled to the second end 128 of the hollow container 122. The connector port 160, as best shown in FIG. 11A, includes a connector receiving region 162 that is adapted for connection to the suction device 100. In particular, as further shown in FIG. 12, the connector interior region 162 is adapted to receive an external portion 102 of the suction device 100.

In certain embodiments, the connector interior region 162 defines an internal opening 163 that is open to, and forms a portion of, the volume 132 of the hollow container 122. The internal opening 163 of the connector interior region 162 is also open with an internal opening 103 of the suction device 100 when the suction device 100 is coupled to the connector port 160, with the internal opening 103 defined within the external portion 102. As such, when negative pressure is actuated (i.e., the suction device 100 is activated) from the suction device 100, suction is created (shown as arrow 200 in FIG. 13) through the internal opening 103, the internal opening 163, and into the volume 132 of the hollow container 122 that is then used that draw the implant 80 (including the shell 82 and gel 84) through the nozzle opening 50 and into the volume 132.

In certain embodiments, such as shown in FIGS. 11 and 11A, the connector port 160 is integrally formed with the second end 128. In these embodiments, the inner surface 164 of the connector receiving region 162, which defines the internal opening 163, extends outwardly from, or otherwise transitions outwardly from the interior container surface 130 of the second end 128 in an opposite direction from the middle portion 124 relative to the nozzle 140. Similarly, the connector receiving region 162 includes an outer surface 166 that extends outwardly from, or otherwise transitions outwardly away from the exterior container surface 134 of the first end 126. The outer surface 166 also includes a depressed region 169 that receives a corresponding flange 107 of the external portion 102 of the suction device 100 to secure the suction device 100 to the connector portion 160.

The connector port 160 also defines a connector axis 165. In particular, the connector axis 165 is defined within the internal opening 163 and is generally aligned parallel to the length of the inner surface 164 of the connector port 160.

In certain embodiments, the connector axis 165 is parallel to the nozzle axis 127 (see FIG. 8B). In further embodiments, the connector axis 165 is offset from the longitudinal axis 125. Still further, in certain embodiments, the connector axis 165 is offset from the longitudinal axis 125 and is radially spaced from the nozzle axis 127 relative to the longitudinal axis 125.

In still further embodiments, the connector port 160 is coupled to the second end 128 and extends in a direction transverse to the longitudinal axis 125. In certain of these embodiments, the connector port 160 is coupled to the second end 128 and extends in a direction normal to the longitudinal axis 125.

In still further embodiments, as opposed to being coupled to the second end 128, the connector port 160 is coupled to the middle portion 124 and extends in a direction transverse to the longitudinal axis 125. In certain of these embodiments, the connector port 160 is coupled to the middle portion 124 and extends in a direction normal to the longitudinal axis 125.

In certain other embodiments, the connector port 160 is a separate structure that is coupled to the second end 128. In certain of these embodiments, the outer surface 166 of the connector port 160 is coupled to the interior container surface 130 of the second end 128, while in further alternative embodiments the inner surface 164 of the connector port 160 is coupled around the exterior container surface 134 of the second end 128. In either of these embodiments, ultrasonic welding or an adhesive material may be introduced between the connector port 160 and the second end 128 to secure the connector port 160 to the first end 128. In embodiments wherein the connector port 160 is coupled to the middle portion 124 and is a separate structure from the middle portion 124, a similar kind of coupling and securing via ultrasonic welding or through an adhesive can occur.

In certain embodiments, the hollow container 122 includes a second cap 170 defining the second end 128. In these embodiments, the second cap 170 is integrally formed with the middle portion 124 as a one-piece structure, such as best shown in FIGS. 11 and 12.

Alternatively, the second cap 170 may be coupled to another terminal end surface (not shown) of the middle portion 124 in a manner similar to the connection of the first cap 129 to the terminal end region 131 of the middle portion 124 as described above.

The hollow container 122, in certain embodiments, is formed from a material having physical properties, particularly in terms of leakage prevention, durability and strength, appropriate for the temporary acceptance of the shell 82, any silicone gel 84, and associated medical waste from the patient 95. Preferably, the hollow container 122 is transparent such that a doctor can confirm the acceptance of the breast implant shell 82, any silicone gel 84, and associated medical waste from the patient 95 during the surgical procedure. In certain embodiments, however, the separate first end 126 (including the nozzle 140) may be formed from an opaque material, such as an opaque plastic material. Still further, as noted above, the material used in the nozzle 140 should have sufficient rigidity to maintain its shape when pressed against the shell 82 of the implant 80 during the removal process. Yet still further, it is desirable that the material used in forming the hollow container 122 has sufficient rigidity and strength to withstand collapsing upon the introduction of negative pressure, in the range of 150-500 mm Hg (mercury), and more preferably from 300 to 500 mm Hg during the removal process. Even still further, it is desirable that such material retain their physical properties, including transparency, after a sterilization process such as through e-beam sterilization.

Illustrative materials for forming the hollow container 122, or any component thereof (such as the nozzle 140), include, but are not limited, thermoplastic materials such as polycarbonate. In certain embodiments, the materials for each separate piece of the hollow container 122 are formed from the same illustrative material, while in other embodiments the separate pieces may be formed of different materials, or slightly different formulations of the same general thermoplastic material. For example, in embodiments, wherein the first end 126 is formed from an opaque material, while the middle portion 124 and second end 128 are formed from a transparent material, the difference in such materials could simply occur by adjusting the formulations to include additives (such as including a fillers or pigments in the first end 126 that are not present, or present in differing amounts, in the middle portion 124 or second end 128). Alternatively, the polymer composition of the thermoplastic materials may be different (such as through the use of a polycarbonate material in one instance and a polyurethane in another instance, or alternatively wherein the polycarbonate material is modified in some other manner (such as through changes in number average molecular weights or by modifying the structure to include additional chemical groups). Other materials, such as thermosetting polymeric materials or glass, may also form one or more of the separate components of the hollow container 122.

In certain embodiments, the coating layer 110 (same as the coating layer 110 described above with reference to FIGS. 8-14) is applied to the interior nozzle surface 144 of the nozzle 140. The coating layer 110 is in the form of a low friction coating layer 110 formed from a low friction material, in which the outer surface 112 (shown in FIG. 12) of the coating layer 110, opposite the interior nozzle surface 144, has lower surface friction than the corresponding surface friction of the interior nozzle surface 144. In certain embodiments, in addition to forming a low surface friction outer surface, the coating material also is a hydrophilic coating, which is believed to provide the outer surface layer with an increased amount of lubricity.

The lower surface friction properties of the outer surface 112 of the coating layer 110, alone or in combination with the increase in hydrophilicity, aids in preventing the implant 80 and any associated residual medical waste (such as blood, tissue, water etc.) from a patient from adhering onto the outer surface 112 during use of implant removal device 121 in removing the implant 80 as compared with an uncoated interior nozzle surface 144, thereby increasing the efficiency of the implant removal process.

In still further embodiments, in addition to having the lower surface friction properties, the outer surface 112 of the coating layer 110 may also provide increased durability properties, or enhanced durability properties, as compared with devices 121 not include such a coating layer 110. Further, the coating layers 110 provided have sufficient bonding strength to the interior nozzle surface 144 such that they are not removed, delaminated, or otherwise degrade during the implant removal process.

Illustrative, non-limiting coating compositions used to form the coating layer 110 include low friction, hydrophilic coating compositions sold under the tradename Serene™, commercially available from Surmodics, Inc. of Eden Prairie, Minnesota and hydrophobic coating compositions sold under the tradename Hydak®, commercially available from Biocoat, Inc., of Horsham, Pennsylvania. However, other coating compositions for forming layers 110 having specific properties or combination of properties are also contemplated and could be used in combination with, or in the alternative to, the coating compositions described above if necessary or desired.

In even further embodiments, a flexible diaphragm may be provided that is positioned at the interface between the hollow container 122 and the suction device 100, or within the interior of the hollow container 122 at a position near, along or adjacent to the connector port 160, to separate the volume 132 of the hollow container 125 from the suction device 100. The flexible diaphragm functions to limit contamination of the suction device 100 from the removed implant 80 during use, the flexible diaphragm also prevents the introduction of cross contamination into the volume 132 of the hollow container 122, and into the patient 95 via the volume 132 of the hollow container 122, from the outside environment. When included, the flexible diaphragm does not adversely impact the generation of negative pressure from the suction device 100 within the volume 132 of the hollow container 122.

Referring now primarily to FIGS. 13-17, with continued reference to FIGS. 8A-12, the present disclosure also is directed to an implant removal system 120, including the afore-mentioned implant removal device 121, and an associated method which is used in a surgical procedure to remove a ruptured silicone breast implant 80 from a patient 95. In the implant removal system 120, as shown in FIG. 13, the implant removal device 121 is coupled to a suction device 100, such as a wall suction device 100 coupled to and extending from a wall 140 within an operating room 150 that when actuated provides negative pressure, in the range of 150-500 mm Hg, within the volume 132 of the hollow container 122 and through the nozzle opening 50 sufficient to facilitate the removal of the shell 82 and silicone gel 84 from the patient 95.

The suction device 100 is conventional in nature and is of the type found in hospital or medical settings to provide a vacuum source for surgical procedures. The suction device 100 may be mounted in an operating room 150 of a medical facility, or alternatively may be mobile.

The subject disclosure is also directed to an associated method for removal a surgical implant 80 from a patient 95, as shown in FIGS. 14-17.

In particular, as best shown in FIGS. 14, 15 and 17, during the surgical procedure for removal on an implant 80, here shown as a ruptured silicone breast implant 80, a small incision 90 is made in the skin of a patient 95 in order to access the ruptured implant 80.

The device 121, which has been inspected and sterilized before use such as through e-beam sterilization as described above and preferably after the nozzle 140 has been briefly dipped into a basin of sterile solution, is then brought into proximity with the incision 90 and is utilized to remove the implant 80. The device 121 is positioned on the surface of the skin near the incision 90 between the ends of the incision 90, with the nozzle opening 50 positioned in closest proximity to the skin of the patient 95 for easier insertion. The nozzle 140 is then inserted within the small incision 90 in the skin and brought into proximity to the implant 80. The size of the small incision 90 is preferably roughly equal to the cross-sectional width of the nozzle 140 such that the nozzle 140 is surrounded by the tissue and skin of the patient 95 after insertion, although the size could be larger if desired based on the preference of the doctor.

As shown clearly in FIG. 14, preferably, during the insertion process, the gripping portions 156 are brought into pressing contact with the shell 82 of the implant 80 such that the nozzle 140 is sealed to the implant 80. As noted above, the rigidity of the nozzle 140 and the gripping portions 156 allows the device 121 to maintain its internal position within the incision 90 of the patient 120 during the implant removal process.

The suction device 100 is coupled to the connector port 160 either prior to the insertion of the nozzle 140 or after the insertion of the nozzle 140. Typically, this is accomplished by inserting the external portion 102 of the suction device 100 into the connector receiving portion 162 and securing the flange 107 into the depressed portion 169 of the outer surface 166.

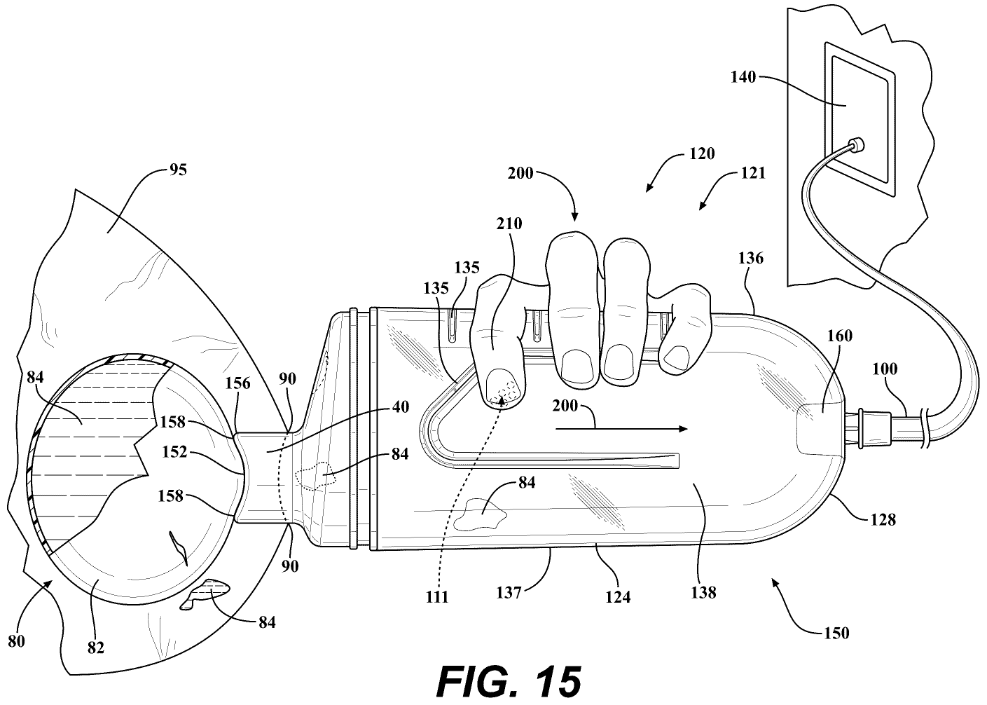

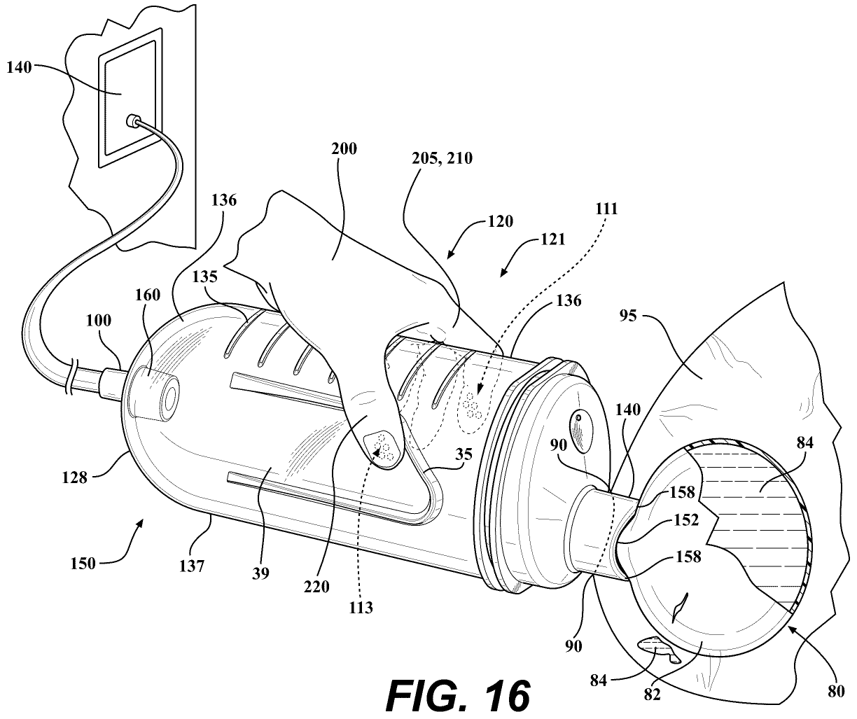

The suction device 100 is then actuated (i.e., activated), thereby creating negative pressure (i.e., vacuum suction/reduced pressure) within the volume 132 of the hollow container 122 (shown by arrow 200 in FIG. 14) sufficient to draw out the breast implant shell 82 and cohesive silicone gel 84 through the nozzle opening 50 of the device 121 and into the volume 132 of the hollow container 122. As illustrated in FIG. 15 and FIG. 16, a portion of the silicone gel 84 has been removed to the volume 132, while the majority of the gel 84 contained in the shell 82 still remains in the patient 95, such as immediately after actuation. As noted above, in certain embodiments, such negative pressure may be between 150 and 500 mm Hg as defined by the system 120 as defined primarily by wall suction device 100. Because the middle portion 124 of the device 121 is transparent, the doctor can observe and confirm the introduction of the shell 82 and silicone gel 84 in the volume 132 of the hollow container 122 during the removal process.

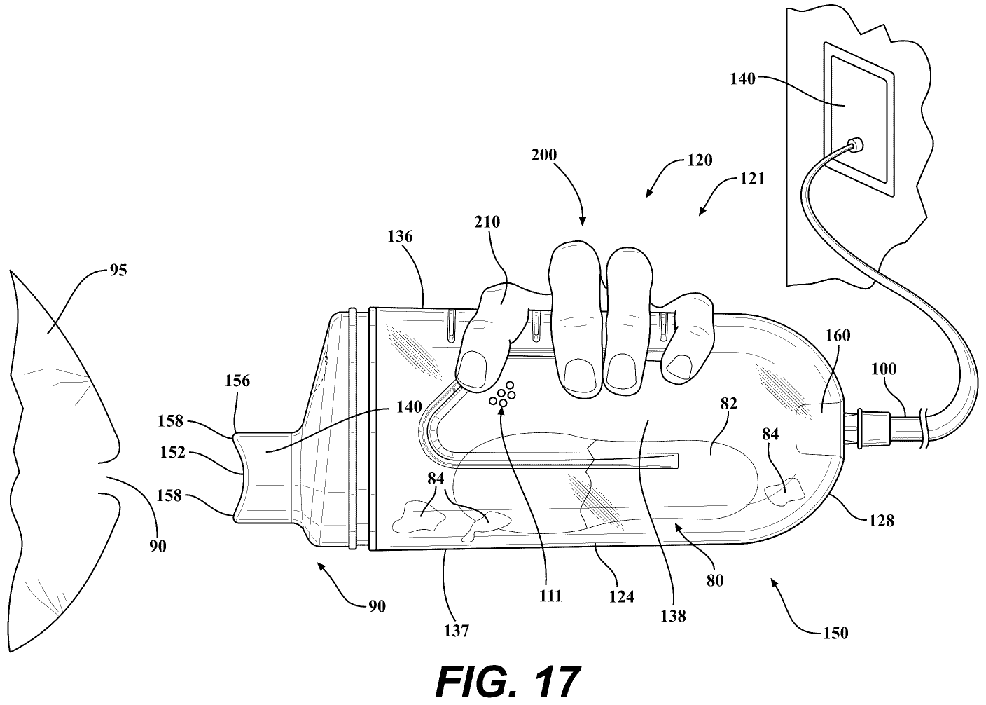

Upon confirmation, typically visual confirmation by the doctor, that a desired amount of the implant shell 82 and silicone gel 84 has been removed from the patient 95 and is visible through the transparent middle portion 124 in the volume 132, the suction device 100 is turned off (i.e., deactivated) and the negative pressure is relieved fully, and the device 121 is removed from the incision 90, as shown in FIG. 17. The suction device 100 is then disconnected from the connector port 160. Any residual silicone gel 84 or shell 82 of the implant 80 that was not removed by the device 121 can then be removed from the patient through the use of surgical sponges or other known manual extraction surgical techniques.

If needed, the negative pressure of the suction device 100 may be increased or decreased relative to a desired range of negative pressure by covering or uncovering the first and/or second set of vent holes 111, 113, as described and illustrated in FIGS. 14-17, for safely removing the implant 80 while minimizing damage to the tissue of the patient 95.

In particular, the negative pressure of the suction device 100 may be defined as varying between a maximum negative pressure when the nozzle 140 is sealed against the implant 80 and wherein each of the first and second set of vent holes 111, 113 present are covered by the one hand 200 of the operator and a minimum negative pressure when the nozzle 140 is sealed against the implant 80 and wherein each of the first and second vent holes 111, 113 present are uncovered by the one hand 200 of the operator. A desired negative pressure for removing the implant 80 may thus be defined as a being a negative pressure that ranges from the maximum negative pressure to the minimum negative pressure, and includes pressures between the maximum negative pressure and the minimum negative pressure when one or more, but less than all, of the first or second set of vent holes 111, 113 are either partially or fully uncovered (i.e., at least one of the first or second set of vent holes 111 or 113 is at least partially covered and at least one of the first or second set of vent holes 111 or 113 is at least partially uncovered).

Accordingly, to precisely control the negative pressure utilized to remove of the implant 80, the following procedure may be utilized. For ease of description, the removal device system of FIG. 13 is utilized in the description of FIGS. 14-17 below.

In particular, the nozzle 140 is inserted within the small incision 90 in the skin and brought into proximity and preferably into sealing contact to the implant 80 prior to activating the wall suction device 100. The operator holding the hollow container 122 with the one hand 200 uncovers each of the first set of vent holes 111 present on the first side 138 of the middle portion 124 by moving the fingers 205, such as the index finger 210, to uncover each of vent holes 111. In addition, the operator holding the hollow container 122 with the same one hand 200 uncovers each of the second set of vent holes 113 present on the second side 139 of the middle portion 124 by moving the thumb to uncover each of vent holes 113. With the vent holes 111, 113 preferably uncovered, the wall device 100 is then activated, creating the minimum negative pressure (during use) in the hollow container 200. If the negative pressure is sufficient, the implant 80 will be sucked into the cavity 132 of the hollow container 122 through the nozzle opening 50.

If the negative pressure is not sufficient to remove the implant 80, the operator may cover one or more the first set of vent holes 111 using one or more of the fingers 205 and/or cover one or more of the second set of vent holes 113 using the thumb 225 on the one hand to increase the pressure.

For example, the user may uncover one or more, but not all, of the first set of vent holes 111 on the first side 138 of the middle portion 124 of the hollow container 122 (such as covering two of the five vent holes 111 present using the index finger 210 as shown in the embodiment FIG. 14) to increase the negative pressure to a desired negative pressure between the minimum negative pressure and the maximum negative pressure.

Alternatively, as shown in FIG. 15, the operator may cover all of the first set of vent holes 111 present on the first side 138 using one or more of the fingers 205 (such as the index finger 210 as shown in FIG. 15) to increase the negative pressure to a desired negative pressure greater than the minimum negative pressure. Assuming that the same number of the second set of vent holes 113 are covered by the thumb 225 on the one hand 200 on the opposing side in FIGS. 14 and 15 (not shown), the negative pressure in the hollow container 122 in FIG. 15 is greater therefore than the negative pressure in the hollow container 122 of FIG. 14. If all of the second set of vent holes 113 are covered by the thumb 225 (or in embodiments wherein there are no vent holes 113), the negative pressure in FIG. 15 is the maximum negative pressure for the system 120.

Still further, and in another alternative as shown in FIG. 16, the operator may cover one or more of the second set of vent holes 113 using the thumb 225 (shown in FIG. 16 as covering all five of the second set of vent holes 113) to increase the negative pressure to a desired negative pressure greater than the minimum negative pressure. Assuming that the same number of the first set of vent holes 111 are covered by the fingers 205 on the one hand 200 on the opposing side as illustrated in FIG. 14 or in FIG. 15 (not shown), the negative pressure in the hollow container 122 in FIG. 16 is greater therefore than the negative pressure in the hollow container 122 of FIG. 14 and greater than the negative pressure in the hollow container 122 of FIG. 15. If all of the first set of vent holes 111 are covered by the fingers (or in embodiments wherein there are no vent holes 111), the negative pressure in FIG. 16 is the maximum negative pressure for the system 120.

In instances where sealing contact with the implant 80 is lost, the operator may uncover the first set of vent holes 111 and/or the second set of vent holes 113 and reposition the device 121 and resume the removal process. Still further, to stop the suction process at any time, operator may uncover the first set of vent holes 111 and/or the second set of vent holes 113, deactivate the wall suction device 100, and remove the device from the incision at any time.

Once the implant 80 has been removed, the operator removes the device 121 from the incision 90 of the patient 95 and deactivates the suction device 100. Preferably, the suction device 100 is deactivated prior to removal. Even still further, in certain embodiments, it is preferable to uncover one or more of the first set of vent holes 111 (see FIG. 17) and/or the one or more of the second set of vent holes 113 prior to the removal of the device 121 from the incision, and in certain instances prior to the deactivation of the suction device 100.

The device 121, including the extravagated silicone gel 84 and shell 82 contained within the volume 132 of the hollow container 122 as best illustrated in FIG. 17, may be disposed of as medical waste. Alternatively, such materials may be removed from the device 121, and the device 121 can be cleaned, sterilized, and reused as desired.

While the device 121 is ideally suited for the removal of silicone breast implants, and in particular ruptured silicone breast implants, the device 121 is also appropriate for use, in the method described above, for removal of other types of implants having a shell and material contained within a shell (such as silicone gel, saline, or some other filling material). In addition, the device 121 is contemplated for use in removing objects that are not implants. By way of non-limiting examples, such objects could include the removal of other substances or objects from an animal or objects from a structure, particularly objects sized and having the flexibility to be drawn through the nozzle opening 140 and into the hollow container 122 at the negative pressures contemplated herein.

Although the present invention and its advantages have been disclosed in the context of certain illustrative, non-limiting embodiments, it should be understood that various changes, substitutions, permutations, and alterations can be made without departing from the scope of the invention as defined by the claims. It will be appreciated that any feature that is described in a connection to any one embodiment may also be applicable to any other embodiment.

What is claimed:

1. A removal device for removing an implant from a patient by an operator, the removal device comprising:
a hollow container extending between a first end and a second end along a longitudinal axis with a middle portion disposed between the first and second ends, and the hollow container having an interior container surface and an opposing, exterior container surface with the interior container surface defining a volume for receiving and containing the implant;
a connector port coupled to the second end of the hollow container and adapted for connection to a suction device;
a nozzle coupled to the first end of the hollow container extending away from the first end along a nozzle axis with the nozzle having an interior nozzle surface and terminating at a nozzle opening; and
wherein the hollow container defines a set of vent holes extending between the interior and exterior container surfaces, and wherein the set of vent holes is sized and configured to be selectively covered by one or more digits of the operator.

2. The removal device of claim 1, wherein the middle portion of the hollow container includes a top side, a bottom side, a first side, and a second side, each side extending between the first end and the second end, and wherein the set of vent holes is positioned on at least one of the first side or the second side.

3. The removal device of claim 2, wherein the set of vent holes is capable of being fully covered by the operator when the operator grasps the hollow container with one hand.

4. The removal device of claim 2, wherein the set of vent holes is capable of being fully covered by a thumb or by a finger on the one hand of the operator when the operator grasps the hollow container with one hand.

5. The removal device of claim 1, wherein the middle portion of the hollow container includes a top side, a bottom side, a first side, and a second side, each side extending between the first end and the second end, and wherein the set of vent holes comprises a first plurality of vent holes and a second plurality of vent holes, and wherein the first plurality of vent holes is positioned on the first side and wherein the second plurality of vent holes is positioned on the second side.

6. The removal device of claim 5, wherein the first plurality of vent holes is configured to be fully covered by a thumb of the one hand of the operator, wherein the second plurality of vent holes is configured to be fully covered by one or more fingers of the one hand of the operator when the operator grasps the hollow container with the one hand.

7. The removal device of claim 1, wherein a size and shape of each of hole of the set of vent holes is the same.

8. The removal device of claim 1, wherein the nozzle opening of the nozzle includes at least one gripping portion and at least one recessed portion for engaging the implant with the nozzle, the nozzle configured to receive the implant through the nozzle opening and into the hollow container, wherein the nozzle axis is offset from the longitudinal axis such that the nozzle opening is offset from the middle portion of the hollow container for aligning with the implant during the removal process, and wherein the at least one gripping portion is aligned proximate the longitudinal axis of the hollow container.

9. The removal device of claim 1, wherein the hollow container is formed from a material having sufficient rigidity to not collapse under normal operating pressures between 150-500 mm Hg.

10. A removal device for removing an implant, the removal device comprising:
a hollow container extending between a first end and a second end along a longitudinal axis with a middle portion disposed between the first and second ends, and the hollow container having an interior container surface defining a volume for receiving and containing the implant;
a connector port coupled to the second end of the hollow container, and adapted for connection to a suction device;
a nozzle, having a cylindrical body, coupled to the first end of the hollow container extending away from the first end along a nozzle axis with the nozzle having an interior nozzle surface and terminating at a nozzle opening, and the nozzle axis being offset from the longitudinal axis such that the nozzle opening is offset from the middle portion of the hollow container along the longitudinal axis for aligning with the implant during a removal process;
a first plurality of vent holes positioned through the middle portion; and
a second plurality of vent holes positioned through the middle portion on an opposing side from the first plurality of vent holes, the first and second plurality of vent holes configured to reduce negative pressure from what a pressure would otherwise be within the hollow container when the suction device is activated.

11. The removal device of claim 10, wherein the hollow container is formed from a material having sufficient rigidity to not collapse under normal operating pressures, wherein the nozzle opening of the nozzle includes at least one gripping portion and at least one recessed portion, wherein the at least one gripping portion is aligned with the longitudinal axis and wherein the nozzle opening is configured to engage the implant with the nozzle configured to receive the implant through the nozzle opening and into the hollow container.

12. A method for removing an implant through an incision in skin of a patient using a suction device and a removal device, the removal device including a hollow container extending between a first end and a second end along a longitudinal axis with a middle portion disposed between the first and second ends, and the hollow container having an interior container surface and an opposing, exterior container surface with the interior container surface defining a volume, a nozzle coupled to the first end of the hollow container extending away from the first end, wherein the hollow container comprises one or more vent holes extending between the interior and exterior container surfaces, the method comprising the steps of:
coupling the suction device to the second end;
grasping the hollow container with one hand of the operator;
inserting the removal device within the incision such that the nozzle opening is in proximity to the implant;
activating the suction device to create a negative pressure within the volume of the hollow container;
adjusting the negative pressure to a desired negative pressure within the volume of the hollow container by adjusting the position of the one hand of the operator to uncover, partially cover or fully cover the one or more vent holes, the desired negative pressure being a negative pressure from a minimum negative pressure to a maximum negative pressure; and
drawing the implant from the patient through the nozzle and into the volume of the hollow container at the desired negative pressure.

13. The method of claim 12, wherein the desired negative pressure is a minimum negative pressure when each vent hole of the one or more vent holes is uncovered by the one hand of the operator, and wherein the desired negative pressure is a maximum negative pressure when each vent hole of the one or more vent holes is covered by the one hand of the operator.

14. The method of claim 12, wherein the desired negative pressure is between the maximum negative pressure and the minimum negative pressure when at least one vent hole from the one or more vent holes is at least partially covered by the one hand of the operator.

15. The method of claim 12, wherein the middle portion of the hollow container includes a top side, a bottom side, and pair of opposing sides, each the side extending between the first end and the second end, and wherein the one or more vent holes are positioned on at least one side of the pair of opposing sides, and wherein the step of adjusting the negative pressure to a desired negative pressure within the volume of the hollow container comprises adjusting the position of the one hand of the operator to uncover, partially cover or fully cover the one or more vent holes positioned on the at least one side of the pair of opposing sides.

16. The method of claim 12, wherein the one or more vent holes comprise a first plurality of vent holes and a second plurality of vent holes, wherein the middle portion of the hollow container includes a top side, a bottom side, a first side, and a second side, each the side extending between the first end and the second end, wherein the first plurality of vent holes is positioned on the first side, wherein the second plurality of vent holes is positioned on the second side, and wherein the step of adjusting the negative pressure to a desired negative pressure within the volume of the hollow container comprises adjusting the position of the one hand of the operator to uncover, partially cover or fully cover one or more of the first plurality of vent holes positioned on the first side, and adjusting the position of the one hand of the operator to uncover, partially cover or fully cover one or more of the second plurality of vent holes positioned on the second side.

17. The method of claim 16, wherein the step of adjusting the negative pressure to a desired negative pressure within the volume of the hollow container comprises:
adjusting the position of a finger on the one hand of the operator to uncover, partially cover or fully cover the first plurality of vent holes positioned on the first side; and
adjusting the position of the thumb of the one hand of the operator to uncover, partially cover or fully cover the second plurality of vent holes positioned on the second side.

18. The method of claim 12 further comprising:
uncovering each of the one or more vent holes after the step of drawing out the implant from the patient through the nozzle and into the volume of the hollow container at the desired negative pressure;
removing the removal device from the incision; and
deactivating the suction device prior to the step of removing the removal device from the incision.

19. The method of claim 12, wherein the step of inserting the removal device within the incision such that the nozzle opening is in proximity to the implant comprises the step of inserting the removal device within the incision such that the nozzle opening is sealed to the implant, and wherein the step of inserting the removal device within the incision such that the nozzle opening is sealed to the implant is done prior to the step of activating the suction device to create a negative pressure within the volume of the hollow container.
